(19) United States
(12) Patent Application Publication
(10) Pub. No.: US 2007/0010818 A1
(43) Pub. Date: Jan. 11, 2007

(54) Surgical system for joints

(76) Inventors: Howard A. Stone, Glenview, IL (US); Larry P. Backes, Libertyville, IL (US)

Correspondence Address: MCANDREWS HELD & MALLOY, LTD, 500 WEST MADISON STREET, SUITE 3400, CHICAGO, IL, 60661, US

(21) Appl. No.: 11/175,653

(22) Filed: Jul. 6, 2005

(51) Int. Cl. A61B 17/58

(52) U.S. Cl. 606/72

(57) ABSTRACT

A surgical kit including a surgical guide system for drilling bones at a joint, a surgical implant for attachment to the bones at the joint, and a surgical implant for attachment to the bones at the joint.

### BACKGROUND OF THE INVENTION

The present invention relates to a surgical fastening system for treatment of a joint. More particularly, certain embodiments of the present invention relate to a surgical fastening system for inserting an implant to treat joint pain and specifically, bunions. The surgical fastening system includes a surgical guide, an implant, and a fastener.

Some joints in the human body carry extreme loads caused by such things as body weight, gait impact, lifting heavy objects, or choice of apparel. In the event these joints are structurally deficient, chronic and progressive subluxation of the joint can occur. A subluxation is a partial dislocation of a bone in a joint. This movement can result in deformity, pain, embarrassment, or other undesirable conditions. There are numerous conditions that are related to such movements at joints.

One of the most common subluxations is Hallux Abducto Valgus (HAV). HAV is a very common and painful foot disorder resulting in the formation of a bunion, recognized as a bony bulge, on the inside of the base of the big toe. HAV occurs when, for complex reasons, the first metatarsophalangeal joint (or MPJ) subluxes the first metatarsal bone medially and the base of the big toe (or Hallux) laterally. This joint movement results in a pronounced and painful bunion that is often embarrassing to the patient, and can lead to other complications and afflictions. There are many contributing factors to HAV. The joint may be deficient in transmitting and distributing the tremendous forces generated by a person's body weight while standing and moving. The condition may be exacerbated by the design of footwear, especially women's high heeled shoes and shoes that form a pointed toe. These types of footwear can shift more of the weight forward onto the ball of the foot, and thus provide even higher lateral forces for the foot's structure to carry. People with flat feet are also more prone to form bunions than those with high arches. Some forms of arthritis can contribute to bunion formation as well. The hereditary condition that predisposes persons to HAV leads to chronic worsening of the condition over time.

A physician measures the severity of HAV by measuring several angles of the foot's bone structure. These angles are seen using an X-ray. The first angle measured is the intermetatarsal (or IM) angle created by lines bisecting the central portions of the first and second metatarsal shafts of the foot. The second angle measured is the Hallux Abductus Angle (or HAA) created by the intersection of lines bisecting the central portions of the first metatarsal and the proximal phalanx of the hallux. FIG. 1 illustrates a top dorsal view of a normal foot 10 including the first metatarsal 14, second metatarsal 18, and first phalangeal bone (Hallux) 22. The IM and HAA 26 and 30 are depicted as well. FIG. 2 illustrates a top dorsal view of a foot 10 afflicted with HAV. A medial eminence 34 of the first metatarsal 14 is particularly evident in its displaced position. The IM and HAA 26 and 30 are clearly greater than those depicted in FIG. 1.

Two other angles (not shown) are useful in the evaluation of HAV. The Proximal Articular Set Angle (P.A.S.A.) is the angular relationship of the bisection of a line through the central portion of the first metatarsal 14 and a line which parallels the articular cartilage of the first metatarsal head. The Distal Articular Set Angle (D.A.S.A.) is the angular relationship of the bisection of a line through the central portion of the proximal phalanx of the hallux 22 and a line perpendicular to the articular cartilage of the base of the proximal phalanx of the hallux.

The IM angle is considered normal from 0-8 degrees. In individuals prone to bunion formation, this angle increases to between 9 and 35 degrees. The normal range for the HAA angle is less than 15 degrees. During bunion formation, the HAA angle can increase to greater than 30 degrees. HAV worsens as the IM and HAA angles increase. Normal P.A.S.A. and D.A.S.A. angles are 0-8 degrees. Every degree of increase of these angles is accompanied by more pain. Conventional treatments of HAV include externally applying pads and cushions to the foot to relieve the growing pain. In addition, anti-inflammatory and pain reducing medications may be used. These methods are only useful at mitigating symptoms and have no affect on the root cause of the condition. For many patients, surgery is required to correct HAV. Hundreds of surgical procedures have been described for correction of HAV. They include simple bunion removal, distal first metatarsal osteotomies, proximal first metatarsal osteotomies, metatarsal-cuneiform joint procedures, and hallux osteotomies. Many of the surgical procedures for HAV attach implants or plates to the bones to limit the movements of bones and joints and allow for healing in the treated area.

There are numerous simple bunions surgeries used to treat HAV. A Silver bunionectomy may be performed when the first MPJ is rectus and there is a normal or low IM. This procedure involves the simple removal of a prominent medial or dorso-medial prominence 34 of bone at the head of the first metatarsal 14 as shown in FIG. 3. A McBride or Modified McBride bunionectomy includes adding a fibular sesmoid release, performing an adductor hallucis tenotomy and/or transfer, or a possibly a fibular sesmoid removal. These procedures are employed when mild abducto valgus of the hallux is present with no or a small increase in the IM angle. These procedures are commonly performed with a first metatarsal osteotomy. A Keller procedure (joint destructive) involves resection of the base of the proximal phalanx of the hallux. Such a procedure is rarely used today due to a multitude of post-operative complications and typically is only performed on older patients with an arthritic bunion.

Hallux osteotomies (known as an Akin or modified Akin procedure) are adjunctive procedures. An Akin is performed when the D.A.S.A. is increased. The procedure involves taking a wedge of bone to re-align the long axis of the proximal phalanx of the hallux. An Akin osteotomy can help re-align the long axis of the EHL tendon more medially to reduce lateral movement of the hallux. The osteotomy can result in slow-healing, especially if the lateral hinge is fractured. This osteotomy is more difficult to fixate especially when a transverse osteotomy line is left. Oblique Akin cuts can be fixated with screws, pins, wires, or staples.

Several types of distal first metatarsal osteotomies have also been developed. These osteotomies are performed when the IM angle is up to 15 degrees. Modifications of these osteotomies, which include wedging and angulating, can correct bi-plane and tri-plane deformities. The most commonly employed osteotomies are the Austin, Modified Austin, Reverdin with modifications, Scarf, Mitchell, and Hohman.

Alternatively, proximal first osteotomies are performed when the IM angle is greater than 15 degrees. These procedures include Opening and Closing base wedge, crescentic, and transverse oblique osteotomies.

Metatarsal-cuneiform procedures are performed when the IM is greater than 15 degrees, a hypermobile first ray is present, and significant metatarsus adductus is present. Such procedures include a Lapidus fusion and cuneiform osteotomies.

Besides HAV, Hallux Limitus (HL) and Hallux Rigidus (HR) are other common conditions of the big toe joint. These conditions are characterized by limited Sagittal plane motion of the MPJ with associated joint compression causing pain, stiffness, and arthritis. Hallux Limitus refers to the earlier stages of first MPJ arthritis where joint motion is “limited”. Hallux Rigidus refers to end stage arthritis where joint motion is “rigid”. These conditions are commonly caused from trauma, a long first metatarsal, and a flexible pes piano valgus deformity with a hypermobile first ray. Surgical procedures for HL and HR can be divided into joint salvage versus joint destructive procedures.

Joint salvage procedures have several goals including reduction in joint tension and mobilization of the plantar joint structures. The most commonly performed procedure for HL is a cheilectomy which involves removal of joint osteophytes with resection of the dorsal portion of the first metatarsal head. Distal metatarsal osteotomies are employed in an attempt to decompress the first MPJ by shortening and/or plantarflexing the first metatarsal head. Joint decompression increases joint motion and improves the structural mechanics of the first MPJ. These procedures include wedge, plantarflexory, and decompression osteotomies. Proximal first metatarsal osteotomies are employed for the more significant metatarsus primus elevatus. These osteotomies attempt to plantarflex the first ray and relax the plantar first MPJ structures. Additionally, decompression osteotomies of the hallux have been attempted in the past without great success.

Joint destructive procedures are purely for salvage of the joint to relieve pain. These include Keller arthroplasty with and without implant, Valenti arthroplasty, and first MPJ fusion.

While all the available surgical techniques provide an immediate correction for HAV, HL/HR, and other joint conditions, none of the surgical techniques presently employed prevents these conditions from reoccurring. At least one in every ten adult bunions recur (Mann R. A., Coughlin M. J. Adult hallux valgus. Surgery of the foot and ankle, vol. 1, 6th ed., St. Louis, CV Mosby, 1992). In fact, bunion recurrence in children can be over 50%. (Gerbert. Complication of Austin Bunionectomy, Journal of Foot Surgery, 1978). While this is a significant number of reoccurrences, it is believed the actual number is much higher. For example, many patients who have had surgery believe that surgery was the last chance for resolving their bunion. Because of this belief, they fail to seek additional treatment and simply live with the recurrent bunion. Also, the patients may choose not to return to the same medical discipline since that discipline did not resolve their bunions with surgery. Instead, the patients seek treatment from other non-conventional practitioners and thereby avoid being counted as a reoccurrence.

Furthermore, bunion surgery often includes the use of surgical fasteners which provide other problems for treating bunions. Surgical fasteners are pins and screws used in bunion surgery, and other forms of surgery, that are made from biocompatible materials. Surgical fasteners are used to affix broken or surgically separated bones together to cause healing to occur in a desired configuration. Also, surgical fasteners may be used to hold surgical implants in place so that the implant can perform its desired function. Surgical fasteners are designed to be inserted into the body during surgeries by either a press fit into a hole that was drilled into the bone or a self-tapping threaded screw. In some cases, a surgically approved adhesive is employed to add more strength to the engagement of the fastener with the bone. Surgical fasteners have to be able to withstand pull-out forces and avoid loosening or walking out during normal patient movements. Depending on the application, pull-out forces can be quite high, resulting in the use of larger diameter fasteners and surgical adhesives to prevent pull-out from occurring. Patients often are required to adjust their lifestyles in order to reduce the pull-out forces on the fasteners and/or implant. When these measures fail and the fasteners still pull out or walk out, revision surgery is required to fix the fasteners or to reset them.

With conventional non-surgical fasteners, pull-out is prevented by capturing the distal end of the fasteners with at least one nut that is larger than the hole the fastener goes through. Surgical fasteners, however, cannot employ a nut on the end of the fastener because the physician would need to make an additional incision in order to capture the end of the fastener. Even if the patient and physician were so inclined to try to capture the distal end of the fastener, there may not be functional space available within the body for the addition of a nut to the end of the fastener. Therefore, surgical fasteners typically use threads, surgical adhesives or a press fit to prevent pull-out. The length of the fastener is chosen so that the distal end of it does not protrude beyond the other side of the bone. When pull-out occurs, another incision for revision surgery is necessary to correct the fastener issue.

Therefore, a need exists for a joint treatment system that reduces the need for repeat surgeries and addresses fastener pull-out and walk out.

### BRIEF SUMMARY OF THE INVENTION

Certain embodiments of the present invention include a fastener for use during surgical procedures having a first member having a first shaft, a second member having a second shaft, and an elastomeric component positioned about at least one of the first and second members. The first shaft of the first member engages and secures the second shaft of the second member thereabout within a bore such that the elastomeric component is compressed between the first and second members and expands to retain the first and second members within the bore.

Certain embodiments of the invention include a surgical implant for attachment to two bones at a joint. The implant includes a first member having a first hole and that is connected to a first bone by a fastener at the first hole. The implant includes a second member having a second hole and that is connected to a second bone by a fastener at the second hole. The second member is configured to receive at least a portion of the first member therein such that the first member slidably moves within the second member as the bones of the joint go through a range of motions.

Certain embodiments of the invention include a surgical guide system for drilling at a surgical site for installation of an implant. The surgical guide includes a base member, at least one clamp for connecting the base member to a surgical site, a first guide member configured to be moved about the base member and including a first drill pilot hole, and a second guide member configured to be moved about the first guide member and including a second drill pilot hole. The first guide member is moved about the base member and secured to a position such that the first drill pilot hole is aligned with a first point at the surgical site and the second guide member is moved about the first guide member to a position such that the second drill pilot hole is aligned with a second point at the surgical site in order to guide a drill into the first and second points.

Certain embodiments of the present invention include a surgical kit for treatment of a joint. The kit includes a surgical guide system for drilling bones at the joint. The surgical guide system includes a base member configured to be connected at the joint, a first guide member configured to be moved about the base member and including a first drill pilot hole, and a second guide member configured to be moved about the first guide member and including a second drill pilot hole. The first and second guide members are moved such that the first and second drill pilot holes are aligned with a first bone and a second bone at the joint, respectively, to guide a drill into the first and second bones. The kit includes a surgical implant for attachment to the two bones at the joint. The implant includes a first implant member connected to the first bone and a second implant member connected to the second bone, the second implant member being configured to receive at least a portion of the first implant member therein such that the first implant member slidably moves within the second implant member as the first and second bones go through a range of motions. The kit includes a fastener for connecting the implant to the joint. The fastener includes a first member having a first shaft and a second member having a second shaft, and an elastomeric component. The first shaft of the first member is positioned within a drilled hole in the first or second bone and engages and secures the second shaft of the second member thereabout such that the elastomeric component is compressed between the first and second members and expands to retain the first and second members within the hole.

Certain embodiments of the present invention include a method for surgically treating a joint condition. The method includes providing a surgical guide having a first guide member with a first pilot hole and a second guide member with a second pilot hole, connecting the surgical guide to the joint, moving the first guide member to a position where the first pilot hole is aligned with a first bone of the joint, moving the second guide member with respect to the first guide member to a position where the second pilot hole is aligned with a second bone of the joint, and using the first pilot hole to drill a first drill hole into the first bone and using the second pilot hole to drill a second drill hole into the second bone.

The method further includes providing an implant having a first implant member with a first hole and a second implant member with a second hole, wherein the second implant member receives at least a portion of the first implant member therein, and providing a plurality of fasteners having a first member, a second member, and an elastomeric component. The method further includes providing a surgical spacer for receiving the fastener and being located between the implant and the bone. For each of the first and second implant members, the method includes assembling the first member of a fastener with the elastomeric component and the second member of a fastener, inserting each of the assembled fasteners into each of the first and second holes of the first and second implant members, a spacer, and into each of the first and second drill holes and pulling the first member of each fastener in a direction out of each drill hole such that the elastomeric component of each fastener expands between the first and second members of the fastener to secure the fastener within each drill hole, and securing the first member of each fastener to the second member of each fastener such that each elastomeric component retains each fastener within each drill hole and retains each of the first and second implant members to each of the first and second bones wherein the first implant member can move telescopingly within the second implant member.

Certain embodiments of the present invention include a method for installing a surgical fastener into a drill hole in a bone. The method includes providing a fastener having a first member, a second member, and an elastomeric component. The method includes assembling the first member of the fastener with the elastomeric component and the second member of the fastener, inserting the assembled fastener into a drill hole in a bone, pulling the first member of the fastener in a direction out of the drill hole such that the elastomeric component of the fastener is compressed and expands between the first and second members of the fastener to secure the fastener within the drill hole, and securing the first member of the fastener to the second member of the fastener such that the compressed elastomeric component retains the fastener within the drill hole.

### BRIEF DESCRIPTION OF SEVERAL VIEWS OF THE DRAWINGS

FIG. 1 illustrates a top dorsal view of a normal human foot.

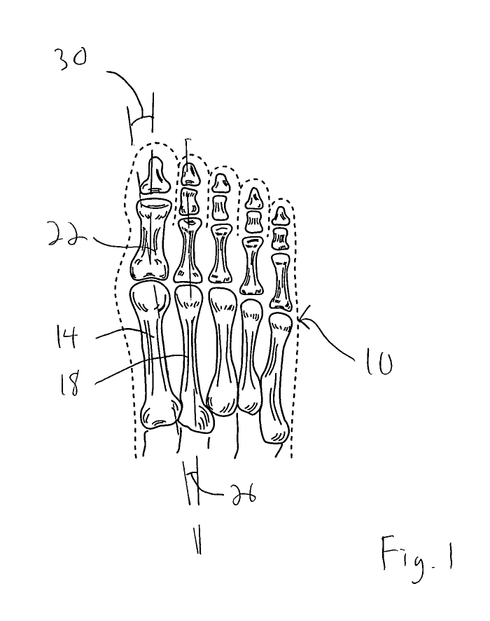

FIG. 2 illustrates a top dorsal view of a foot afflicted with HAV.

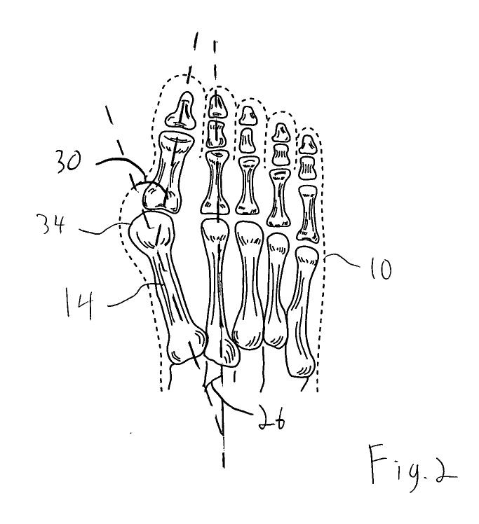

FIG. 3 illustrates a top dorsal view of an HAV-afflicted foot that has been surgically modified using a simple bunionectomy and osteotomy.

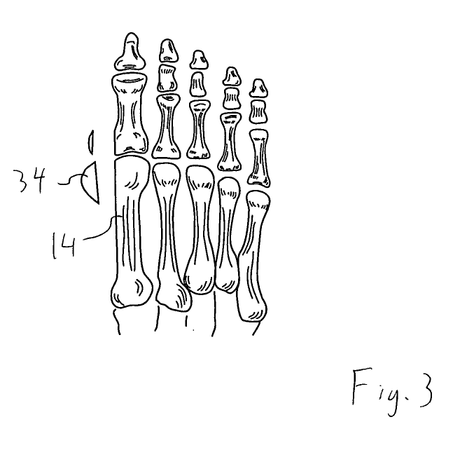

FIG. 4 illustrates an isometric view of an implant according to an embodiment of the present invention.

FIG. 5 illustrates an isometric view of the implant of FIG. 4 in a mated position according to an embodiment of the present invention.

FIG. 6 illustrates an side view of an implant fastener according to an embodiment of the present invention.

FIG. 7 illustrates an isometric view of an external member of the fastener of FIG. 6.

FIG. 8 illustrates an isometric view of an internal member of the fastener of FIG. 6.

FIG. 9 illustrates a front view of the external member of FIG. 7.

FIG. 10 illustrates a rear view of the external member of FIG. 7.

FIG. 11 illustrates a top view of an elastomeric component of the fastener of FIG. 6.

FIG. 12 illustrates a side view of the elastomeric component of FIG. 11.

FIG. 13 illustrates a side view of an engagement tool according to an embodiment of the present invention.

FIG. 14 illustrates a side view of the engagement tool of FIG. 13 engaging the fastener of FIG. 6 before the fastener is activated.

FIG. 15 illustrates a side view of the engagement tool of FIG. 13 engaging the fastener of FIG. 6 after the fastener is activated.

FIG. 16 illustrates a side view of the activated fastener of FIG. 15 receiving a screw cap.

FIG. 17 illustrates a front view of a surgical guide used on a human foot according to an embodiment of the present invention.

FIG. 18 illustrates a side view of the surgical guide of FIG. 17 without the vertical or horizontal members connected.

FIG. 19 illustrates a top view of the surgical guide used on a human foot of FIG. 17.

FIG. 20 illustrates a front isometric view of a vertical member of the surgical guide of FIG. 17.

FIG. 21 illustrates an isometric view of a key of the surgical guide of FIG. 17.

FIG. 22 illustrates a front view of a horizontal member of the surgical guide of FIG. 17.

FIG. 23 illustrates a top view of a spacer formed according to an embodiment of the present invention.

FIG. 24 illustrates an isometric view of the implant of FIG. 5 used with the fastener of FIG. 6 according to an embodiment of the present invention.

FIG. 25 illustrates an isometric view of the implant of FIG. 5 used with the fastener of FIG. 6 according to an embodiment of the present invention.

FIG. 26 illustrates a top view of the implant and fastener of FIG. 25.

The foregoing summary, as well as the following detailed description of certain embodiments of the present invention, will be better understood when read in conjunction with the appended drawings. For the purpose of illustrating the invention, there is shown in the drawings, certain embodiments. It should be understood, however, that the present invention is not limited to the arrangements and instrumentality shown in the attached drawings.

### DETAILED DESCRIPTION OF THE INVENTION

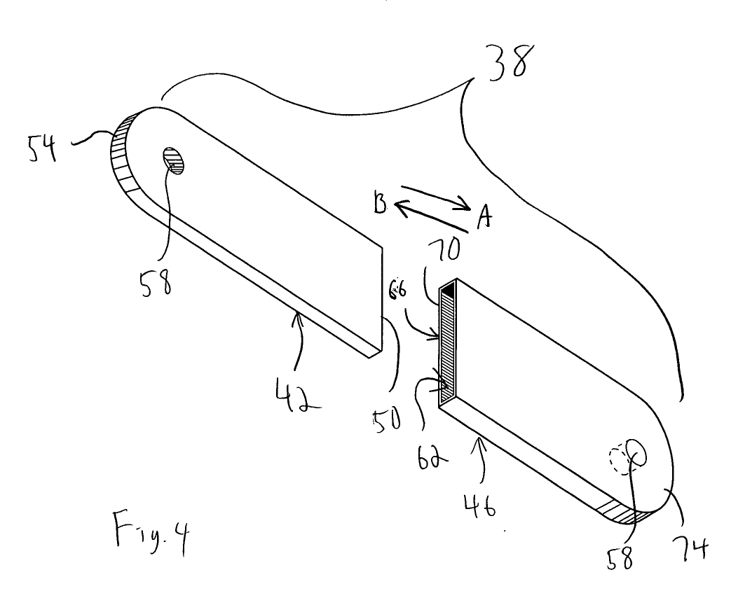

FIG. 4 illustrates an isometric view of an implant 38 according to an embodiment of the present invention. The implant 38 is surgically fastened to bone joints to limit lateral movement and allow longitudinal movement. The implant 38 includes an insertion plate member 42 and an reception plate member 46. The members 42 and 46 are made from any number of hard, rigid, strong materials suitable for implantation into the human body. The insertion member 42 is planar with a straight distal or first end 50 and a curved second end 54. The insertion member 42 has a hole 58 proximate the second end 54 configured to accept a surgical fastener. The reception member 46 is a casing including a bore 62 that extends therethrough from a mouth 66 at a first end 70 to a closed, curved second end 74. The reception member 46 has a hole 58 proximate the second end 74 configured to accept a surgical fastener. The bore 62 is sized slightly larger than, and is of generally the same geometry as, the insertion member 42 in order receive the insertion member 42 therein.

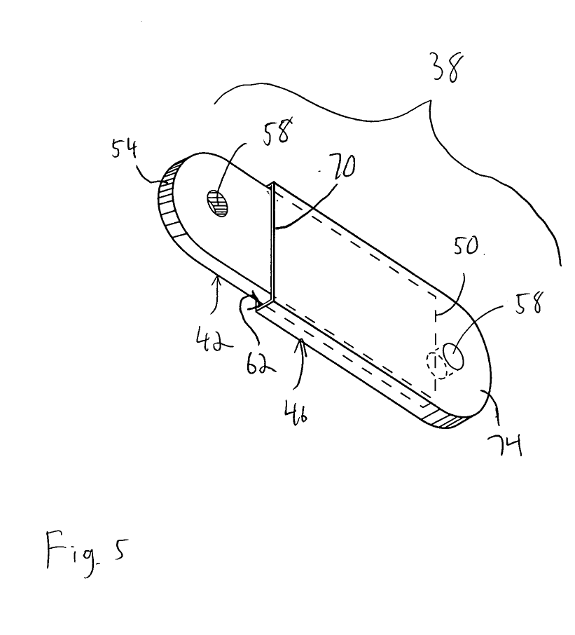

In order to connect the members 42 and 46, the first end 50 of the insertion member 42 is slidably moved in the direction of arrow A into the mouth 66 of the reception member 46 and through the bore 62 until the first end 50 is just at the hole 58 of the reception member 46, as shown in FIG. 5. In operation, the length of the insertion member 42 is pre-determined such that when fully inserted into the reception member 46, the first end 50 of the insertion member 42 contacts the shaft of the surgical fastener received within the hole 58 of the reception member 46. In other words, the first end 50 of the insertion member 42 contacts the shaft of the surgical fastener to provide a precise minimum allowable distance between the heads of each of the bones comprising the joint to which the implant 38 is connected. Once fully inserted into the reception member 46, the insertion member 42 is also movable in the direction of arrow B to move further out of the reception member 46.

The insertion and reception members 42 and 46 have appropriate lengths and widths for use with the particular size of the joint that the implant 38 will stabilize. The members 42 and 46 have depths pre-determined by the strength of the materials chosen and the specific lateral bending forces the mated members 42 and 46 need to resist without interfering with the relative sliding position of the insertion member 42 to the reception member 46. The curved ends 54 and 74 of the members 42 and 46, respectively, permit axial motion at a joint without interference from surrounding soft tissues. The members 42 and 46 may be coated with materials suitable for implantation into the human body that add lubricity and anti-wear properties to the material of the reception member 46.

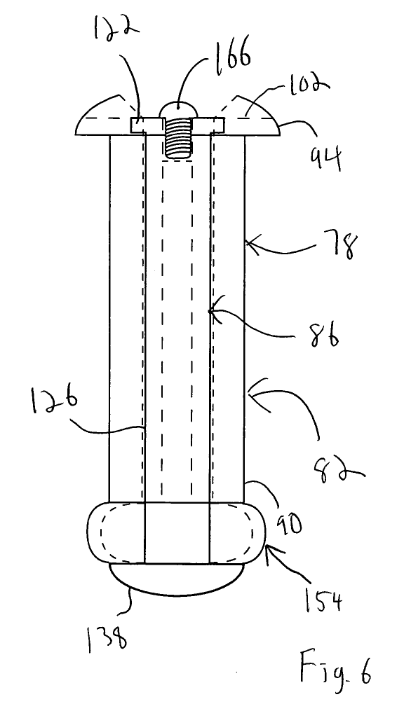

FIG. 6 illustrates an side view of an implant fastener 82 according to an embodiment of the present invention. The implant fastener 82 includes an internal member 86, an external member 78, an elastomeric component 154, and a screw 166. Two surgical fasteners 82 are used to secure the mated members 42 and 46 of FIGS. 4 and 5 to the bones of the joint being treated. Each hole 58 in the members 42 and 46 receives one fastener 82. The fasteners 82 engage the bone and the implant 38 such that large lateral forces against the mated members 42 and 46 do not cause pull-out and such that the axial rotation of each member 42 and 46 about a fastener 82 does not cause the fasteners 82 to walk-out.

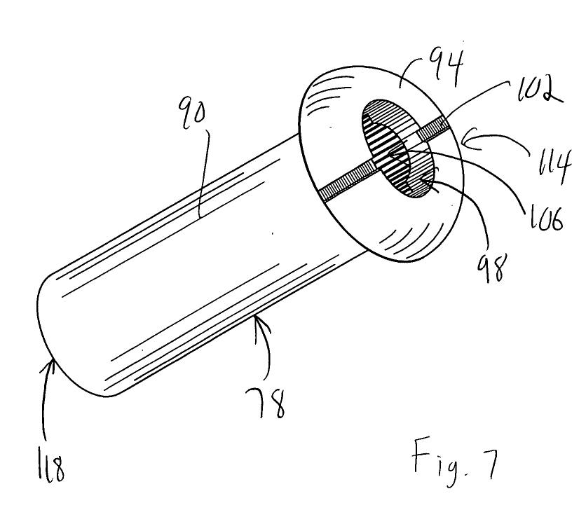

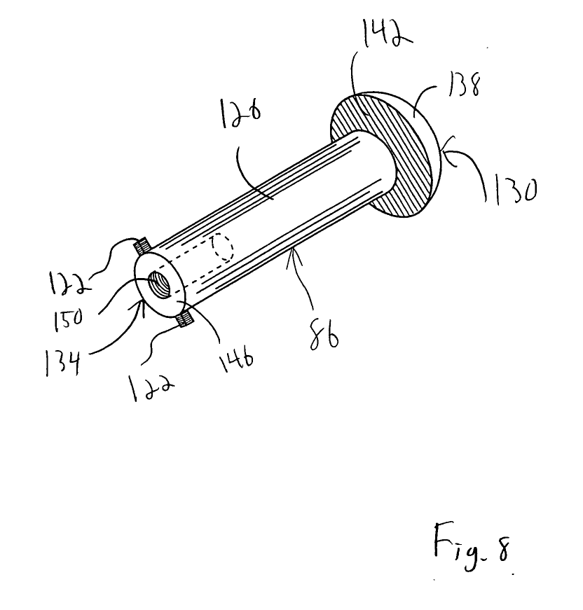

FIG. 7 illustrates an isometric view of the external member 78 of the implant fastener 82 and FIG. 8 illustrates an isometric view is of the internal member 86 of the implant fastener 82. Both the internal member 86 and external member 78 of the fastener 82 are made from hard and strong materials that are suitable for implantation into the human body. The external member 78 includes a cylindrical shaft 90 having a first end 114 and a second end 118. The shaft 90 is formed with a head 94 at the first end 114. The shaft 90 may be coated with materials that would resist or encourage bone growth thereto. The rounded head 94 is of greater diameter than the shaft 90 and has a round aperture 98 in a center region thereof. The head 94 further includes a slot 102 that runs across the center thereof. In operation, when the external member 78 is inserted into a hole 58 of either member 42 or 46 of FIGS. 4 and 5 and into the bone of the joint, the head 94 holds the member in place against the bone. The length of the shaft 90 is generally the same as the depth of the bone through which it is inserted. The external member 78 includes a bore 106 that extends from the aperture 98 along the entire length of the shaft 90. By way of example only, the bore 106 is circular in shape.

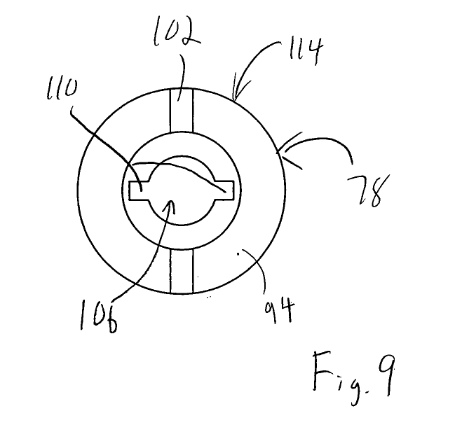

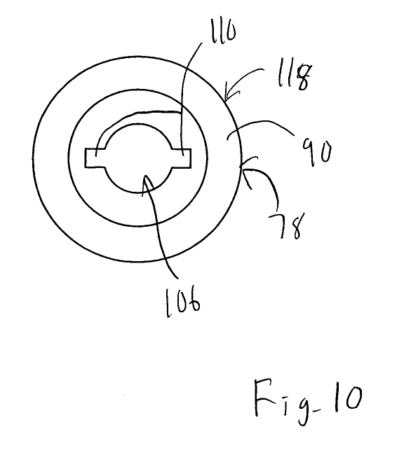

FIG. 9 illustrates a front view of the external member 78 of FIG. 7 at the first end 114 and FIG. 10 illustrates a rear view of the external member 78 of FIG. 7 at the second end 118. Referring to FIG. 9, the external member 78 includes broached slots 110 extending along the bore 106 and located approximately ninety degrees in relation to the slot 102 in the head 94. Referring to both FIGS. 9 and 10, the broached slots 110 run the length of the bore 106 from the first end 114 to the second end 118 and are located approximately 180 degrees opposite of each other on sides of the bore 106. The broached slots 110 have a geometry and depth to slidably receive tabs 122 of the internal member 86 (FIG. 8) without affecting the structural integrity of the shaft 90. The external member 78 may be coated with materials suitable for use inside the human body that add lubricity and anti-wear properties to the hard and strong material of the external member 78.

Referring to FIG. 8, the internal member 86 of the fastener 82 includes a cylindrical shaft 126 having a first end 130 and a second end 134. The shaft 126 is formed with a rounded head 138 at the first end 130. The internal member 86 is made of the same hard and strong material as the external member 78. The diameter of the shaft 126 of the internal member 86 is chosen to be slightly smaller than, and thus receivable within, the bore 106 of the external member 78 (FIG. 7). The head 138 is widest at a base 142. The base 142 has a diameter that is the same as, or slightly smaller than, the outer diameter of the shaft 90 (FIG. 7) of the external member 78. The internal member 86 includes two tabs 122 protruding outwardly from, and perpendicular to, the outer diameter of the shaft 126 and flush with a base 146 of the shaft 126 at the second end 134. The tabs 122 are located approximately 180 degrees opposite of each other, and are of generally the same geometry as the broached slots 110 (FIG. 7) extending along the length of the bore 106 of the external member 78 (FIG. 7). The tabs 122 are dimensioned slightly smaller than the broached slots 110 such that the tabs 122 may be slid within the broached slots 110 along the bore 106 of the external member 78 (FIG. 7) into the slot 102 of the head 94 (FIG. 7). The internal member 86 also includes a circular threaded hole 150 extending internally into the shaft 126 from the second end 134. The threaded hole 150 has a nominal depth to receive an engagement tool and a screw cap.

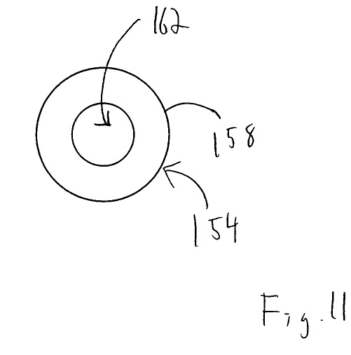

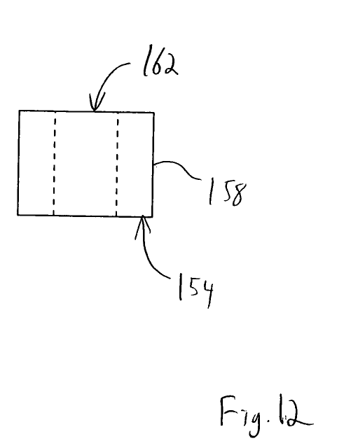

FIGS. 11 and 12 illustrate a top and side view of the elastomeric component 154, respectively. The elastomeric component 154 is cylindrical in shape with a ring-shaped outer wall 158 surrounding a centrally located bore 162. Alternatively, the elastomeric component 154 may have other geometries. The diameter of the bore 162 is slightly larger than the outer diameter of the shaft 126 of the internal member 86 (FIG. 8). The elastomeric component 154 is formed of an elastomeric material that is durable and implantable in the human body. The elastomeric material has a durometer (not shown) associated with it that provides resistance when the elastomeric component 154 is compressed such that the elastomeric component 154 bulges radially outward. The elastomeric component 154 is positioned about the shaft 126 of the internal member 86 (FIG. 6) between the head 138 of the internal member 86 and the shaft 90 of the external member 78 (FIG. 6). In a relaxed state, the elastomeric component 154 has approximately the same outer diameter as that of the shaft 90 of the external member 78 and the head 138 of the internal member 86. Referring to FIG. 6, the length of the elastomeric component 154 is such that when the tabs 122 of the internal member 86 are engaged within the slot 102 of the head 94 of the external member 78, the elastomeric component 154 is flexed and bulging so that its flexed diameter is greater than the outer diameter of the shaft 90 of the external member 78. However, the length of the elastomeric component 154 is small enough so as to not interfere with other structures in proximity to the bone to which the fastener 82 is affixed.

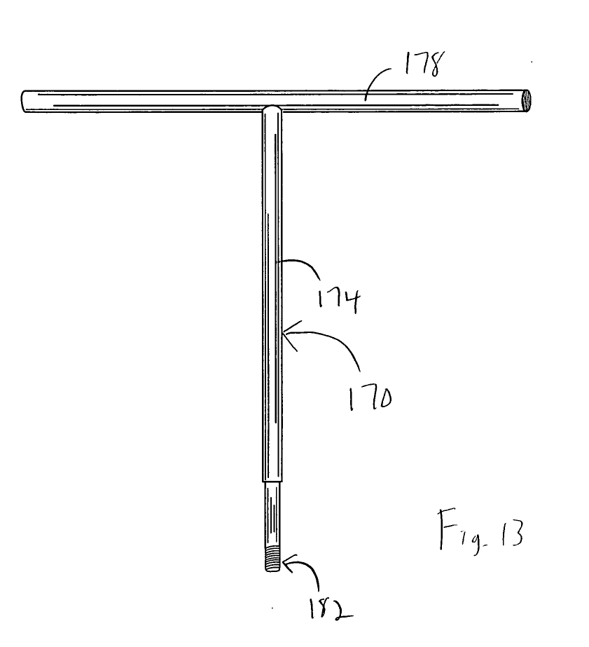

FIG. 13 illustrates a side view of an engagement tool 170 according to an embodiment of the present invention. The engagement tool 170 includes a shaft 174 perpendicularly joined with the center section of a crosspiece 178 to form a “T” shaped handle. By way of example only, the shaft 174 may be welded to the crosspiece 178. The engagement tool 170 is made from a hard and strong material. The shaft 174 includes a threaded tip 182 for engaging the threaded hole 150 (FIG. 8) in the shaft 126 of the internal member 86.

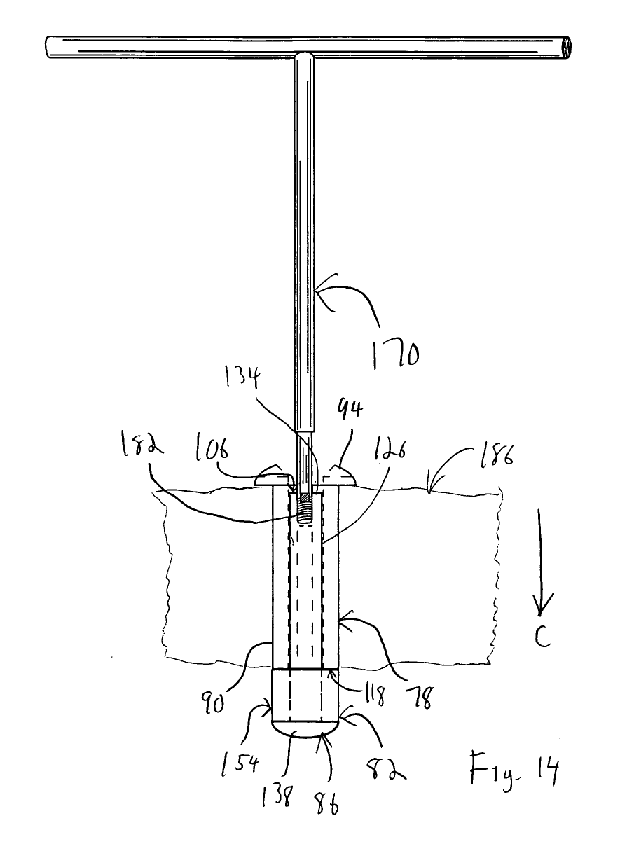

FIG. 14 illustrates a side view of the engagement tool 170 engaging the fastener 82. In operation, the implant fastener 82 is used as follows. During surgery the physician drills a hole through a bone 186 at the joint location where the physician wishes to locate the fastener 82. The diameter of the hole is approximately the same as the outer diameter of the shaft 90 of the external member 78. The length of the fastener 82 is predetermined based on the depth of the bone 186 receiving the fastener 82. The fastener 82 is assembled by positioning the elastomeric component 154 over the shaft 126 of the internal member 86 until the elastomeric component contacts the head 138 of the internal member 86. The physician then inserts the second end 134 of the internal member 86 into the external member 78 such that the bore 106 of the external member 78 slidably receives the shaft 126 of the internal member 86 therein and the broached slots 110 (FIG. 9) receive the tabs 122 (FIG. 8). The physician then inserts and threadably engages the tip 182 of the engagement tool 170 into the threaded hole 150 (FIG. 8) of the internal member 86. Alternatively, the fastener 82 may be provided to the physician preassembled. The physician inserts the assembled fastener 82 into the drilled hole in the direction of arrow C with the head 138 of the internal member 86 going into the hole first. As the physician inserts the fastener 82 into the hole, the physician pushes the external member 78 through the bone 186 by pushing against the head 94 of the external member 78. The fastener 82 is fully inserted when the elastomeric component 154 and the head 138 of the internal member 86 protrude past the other side of the bone 186.

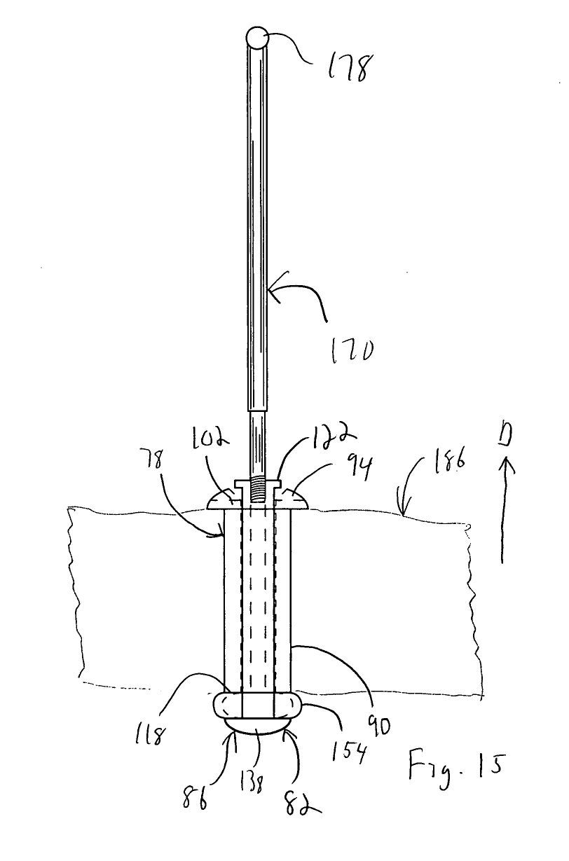

FIG. 15 illustrates a side view of the engagement tool 170 engaging the fastener 82. While holding the head 94 of the external member 78, the physician then pulls the crosspiece 178 of the engagement tool 170 in the direction of arrow D until the tabs 122 of the internal member 86 extend outward beyond the head 94 of the external member 78. Simultaneously, the head 138 of the internal member 86 is pulled in the direction of arrow D to squeeze or compress the elastomeric component 154 between the head 138 and the second end 118 of the external member 78 or the bone 186. As the elastomeric component 154 is compressed, the elastomeric component 154 flexes radially outward to an outer diameter that is greater than the outer diameter of the shaft 90 of the external member 78. The physician then turns the cross piece 178 of the engagement tool 170 approximately ninety degrees to align the tabs 122 of the internal member 86 with the slot 102.

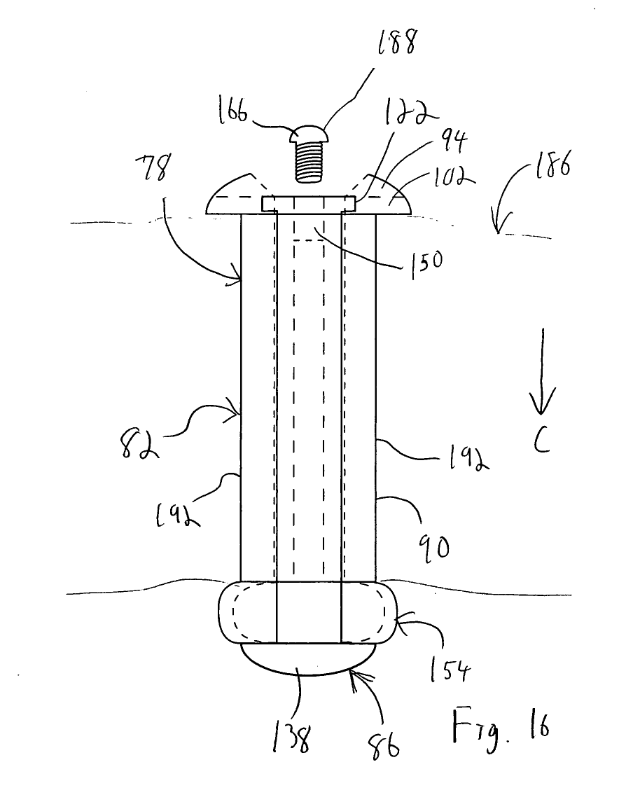

Referring to FIG. 16, the physician then uses the engagement tool 170 (FIG. 15) to move the internal member 86 in the direction of arrow C until the tabs 122 come to rest in the slot 102 of the external member 78. The internal member 86 is longer than the external member 78 such that when the tabs 122 are positioned in the slot 102, the elastomeric component 154 is maintained in a compressed state where the elastomeric component 154 extends outwardly to an outer diameter that is greater than the outer diameter of the shaft 90 of the external member 78. The flexed elastomeric component 154 extends between the bone 186 and the head 138 of the internal member 86 in order to prevent the fastener 82 from being pulled out or walking out of the drilled hole. The engagement tool 170 is then disengaged from the threaded hole 150 of the internal member 86. The tabs 122 of the internal member 86 resting inside the slot 102 do not interfere with disengaging the engagement tool 170 from the threaded hole 150. To close up the threaded hole 150, the physician then threads the cap screw 166 into the threaded hole 150 of the internal member 86 until the cap screw 166 is seated flush with the head 94 of the external member 78.

The geometry of the cap screw 166 has a rounded head 188 to provide a consistent, smooth geometry with the head 94 of the external member 78. The cap screw 166 is made from materials that are implantable into the human body. The screw cap 166 covers up the threaded hole 150 in order that bone or tissue does not grow in the threaded hole 150 in order that the engagement tool 170 can easily be used to engage the threaded hole 150 remove the fastener 82 if necessary. The screw cap 166 may have a slot in the head 188 to allow a tool to engage and disengage the screw cap 166 from the threaded hole 150.

Should the physician choose to later remove the fastener 82, the physician may simply unscrew the cap screw 166, use the engagement tool 170 (FIG. 15) to disengage the tabs 122 of the internal member 86 from the slot 102 of the external member 78 to decompress the elastomeric component 154, and pull the entire fastener 82 out of the bone. In the event that bone growth has occurred on the elastomeric component 154 and internal member 86, the external member 78 may still be removed from the bone 186.

In an alternative embodiment, the fastener 82 may be used in instances where the physician does not drill all the way through the bone 186. In such situations, the fastener 82 may be inserted such that the elastomeric component 154 bulges against walls 192 of the hole in the bone 186 and provides lateral resistance to pull-out or walk-out of the fastener 82. Additionally, the elastomeric component 154 may include coatings and materials that promote bone growth around the elastomeric component 154 and thus aid in anchoring the elastomeric component 154 further in place over time.

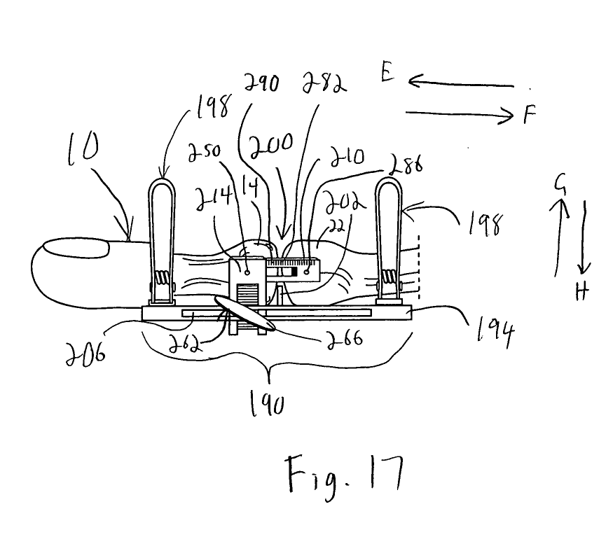

FIG. 17 illustrates a front view of a surgical guide 190 used on a foot 10 as an aid to surgical drilling of holes into the foot 10 at a joint 200. The surgical guide 190 includes a planar base 194 having spring clamps 198 mounted at distal ends thereof and extending perpendicularly from the base 194. The spring clamps 198 are mounted to the base 194 such that the physician may loosen the spring clamps 198 from the base 194 and laterally slide the spring clamps 198 along the base 194 in the directions of arrows E or F to a desired location. The surgical guide 190 includes a spacer gauge 202 that extends upward and inward from the base 194 towards the joint 200. The surgical guide 190 also includes a vertical member 214 that is slidably mounted within a slot 206 in the base 194 and that carries a horizontal member 210 generally parallel to the base 194.

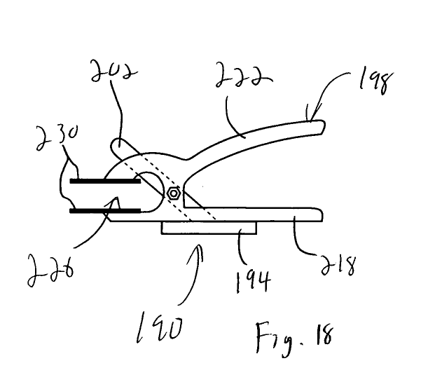

FIG. 18 illustrates a side view of the surgical guide 190 without the vertical or horizontal members 214 or 210 connected and unattached to the foot. Each clamp 198 includes a base handle 218 connected to the base 194 opposite an open handle 222. Each clamp 198 further includes a mouth 226 having oppositely aligned grip pads 230. In operation, the handles 194 and 198 are pivoted relative to each other to open and close the mouth 226, and thus secure the grip pads 230 about a bone. The spacer gauge 202 extends diagonally outward from the base 190 between the clamps 198.

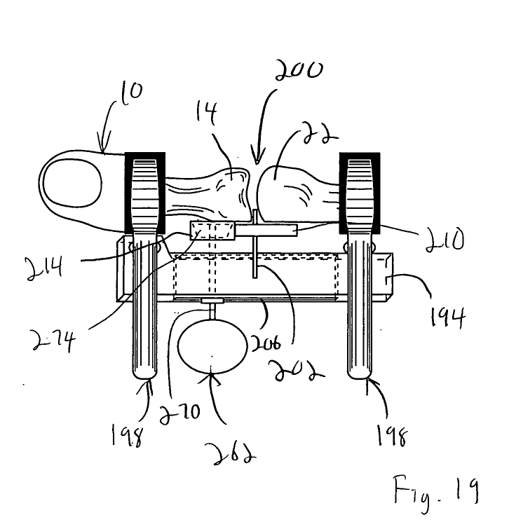

Returning to FIG. 17, in order to connect the surgical guide 190 at the joint 200, the physician uses the spacer gauge 202 to correctly space the bones 14 and 22 of the joint. The width of the spacer gauge 202 is set to the exact minimum distance required to properly separate the heads the bones 14 and 22 at the joint 200. Referring to FIG. 19, the physician places the spacer gauge 202 between the heads of the joint bones 14 and 22. While the spacer gauge 202 is held against the head of one of the joint bones 14 and 22, the physician moves one spring clamp 198 along the base 194 into position and tightens the spring clamp 198 against the shaft of that same joint bone. Then the head of the opposite joint bone can be pushed against the other side of the spacer gauge 202 and the other spring clamp 198 is moved into position and tightened against the shaft of the same joint bone. The joint 200 is held stable by the spring clamps 198 across the base 194, and the heads of the joint bones 14 and 22 are separated by a required minimum distance by use of the spacer gauge 202. When attached across a joint, the base 194 is medial to the joint 200 and below the joint 200 so as to provide stability and an open area in which the physician can work.

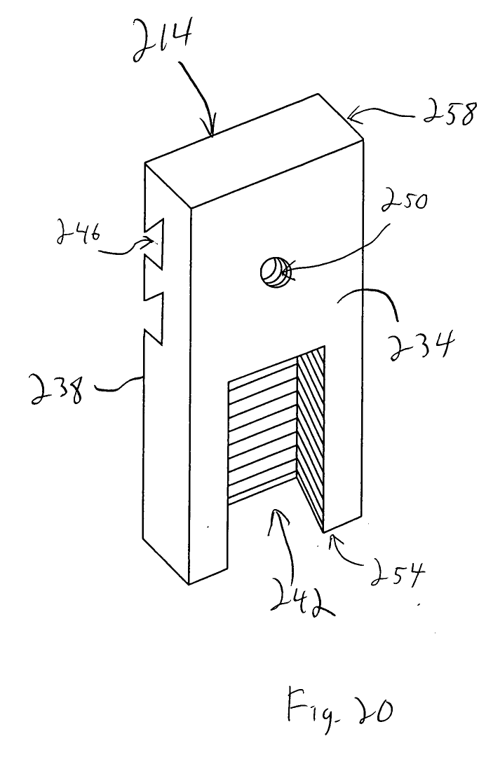

The assembly of the vertical and horizontal members 214 and 210 can be adjusted and easily mounted to the base 194. The vertical member 214 is connected to the base 194 by a key 262 that extends through the slot 206 in the base 194. FIG. 20 illustrates a front isometric view of the vertical member 214 of the surgical guide 190 of FIG. 17. The vertical member 214 is generally planar in shape and has a front side 234 and a rear side 238. The front side 234 includes a vertically oriented dovetail groove 242 extending therein to a bottom end 254. The rear side 238 includes at least one horizontally oriented dovetail groove 246 extending therein and across the length thereof. The vertical member 214 also includes a pilot hole 250 extending therethrough proximate a top end 258. The pilot hole 250 is located so as to not interfere with horizontal dovetail groove 246.

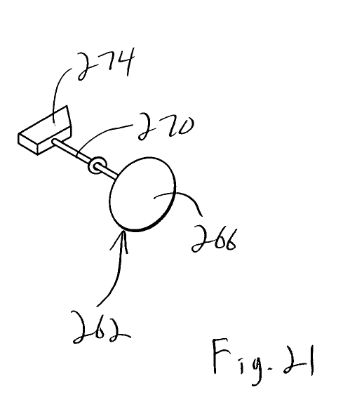

FIG. 21 illustrates an isometric view of the key 262. The key 262 includes a circular handle 266 formed with a cylindrical shaft 270 and a block shaped engagement piece 274. Referring to FIG. 19, the engagement piece 274 is received in the vertical groove 242 (FIG. 20) of the vertical member 214 and the shaft 270 is received in the slot 206 of the base 194. Referring to FIG. 17, the vertical member 214 may moved laterally along the base 194 to a desired position by moving the handle 266 of the key 262 in the directions of arrows E or F. The key 262 is configured to then be locked in place such that the vertical member 214 does not freely move laterally once in the desired position. Also, the vertical member 214 may be moved vertically in the directions of arrows G or H by moving the vertical member 214 about the engagement piece 274 (FIG. 21) of the key 262. Once the vertical member 214 is in the desired vertical position with the pilot hole 250 aligned with the head of the bone 14, the key 262 is configured to function like a thumb screw such that the physician can use the handle 266 to lock the engagement piece 274 within the vertical groove 242 (FIG. 20) of the vertical member 214 and retain the vertical member 242 in a fixed position about the engagement piece 274.

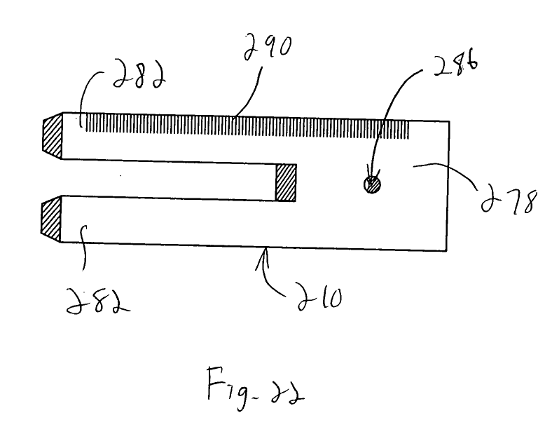

FIG. 22 illustrates a front view of the horizontal member 210 of the surgical guide 190 of FIG. 17. The horizontal member 210 is generally planar in shape and has a body 278 and at least one leg 282 extending from the body 278 that are configured to be slidably received in the grooves 246 (FIG. 20) of the vertical member 214 (FIG. 20). Alternatively, the horizontal member 210 and vertical member 214 may be configured to move with respect to each other by any number of other connection features. The horizontal member 210 includes a pilot hole 286 in the body 278. One of the legs 282 of the horizontal member 210 has distance markings 290 thereon. Referring to FIG. 17, the pilot holes 250 and 286 of the vertical and horizontal members 214 and 210, respectively, are positioned such that when the legs 282 of the horizontal member 210 are engaged within the grooves 246 (FIG. 20) of the vertical member 214, both pilot holes 250 and 286 are in a plane generally parallel to the base 194. The horizontal member 210 may be moved in the direction of arrows E or F with respect to the vertical member 214 to a position where the pilot hole 286 is aligned with the head of the bone 22 and may be retained to the vertical member 214 in a fixed position by a clip, pin, or any number of other securing devices (not shown).

In operation, the implant 38, fasteners 82, and surgical guide 190 may all be used together as part of a surgery kit or system to perform surgery on a bunion or any number of other joint or bone conditions. Referring to FIG. 19, first, the physician makes an incision on the foot 10 along the joint 200 to be treated and pulls back the skin to reveal the joint 200. The surgeon then cuts the bones 14 and 22 as necessary such that they are generally coplanar. Referring to FIG. 17, the surgical guide 190 is connected to the joint 200 by using the spacer gauge 202 and the clamps 198 as described above to secure the surgical guide 190 to the joint 200 in the desired position. The vertical and horizontal member 214 and 210 are adjusted along the joint 200 and locked into desired positions as described above such that the pilot holes 250 and 286 are located at optimal positions on the heads of the bones 14 and 22. The pilot hole 250 of the vertical member 214 may be aligned with the head of the bone 14 and the pilot hole 286 of the horizontal member 210 may be aligned with the head of the bone 22. The relative distance of the pilot hole 286 in the horizontal member 210 to the pilot hole 250 of the vertical member 214 when the spacer gauge 202 is in place between the heads of the bones 14 and 22 determines the exact size of the implant 38 necessary to maintain stability of the joint 200. Where the markings 290 on the horizontal member 210 intersect the vertical member 214 indicates the correct size of the implant 38 the physician should use. When the physician has each pilot hole 250 and 286 located precisely along the heads of the bones 14 and 22 of the joint 200, the physician uses the pilot holes 250 and 286 to drill through the bones 14 and 22 of the joint 200 in preparation of installing the implant 38. By way of example, holes are drilled in the first metatarsal bone 14 and in the first phalangeal bone 22.

Once the physician has finished using the pilot holes 250 and 286 to make properly-spaced and aligned holes in the bones 14 and 22, the physician may easily remove the horizontal and vertical members 210 and 214. The physician unlocks the horizontal member 210 from the vertical member 210 and slides the legs 282 of the horizontal member out of the vertical member 214. The physician then unlocks the vertical member 214 from the engagement piece 274 of the key 262 and slides the vertical member 214 off of the engagement piece 274. The physician then removes the key 262 from the slot 206 in the base 194. Thus, the physician has space for installing the implant 38 while maintaining the joint in a stabilized state by keeping the base 194, spacer gauge 202, and spring clamps 198 in position about the joint 200.

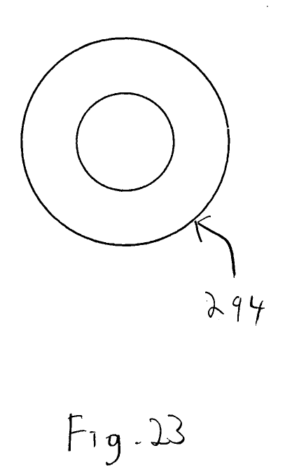

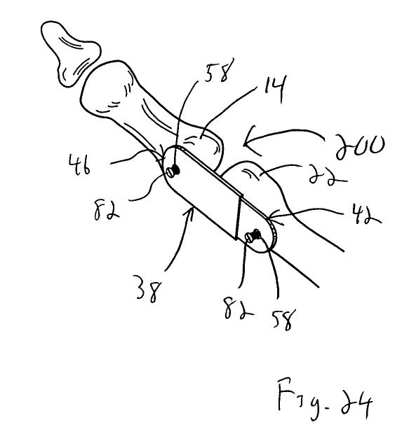

Referring to FIG. 24, the implant 38 is positioned about the joint 200 with the insertion member 42 having a surgical fastener 82 inserted through the hole 58 of the insertion member 42 and mated within the reception member 46 having a surgical fastener 82 inserted through the hole 58 of the reception member 46. The base 194, spacer gauge 202, and spring clamps 198 are not shown in FIG. 24. By way of example only, the hole 58 of the reception member 42 is aligned with the hole in the first metatarsal bone 14 and the hole 58 of the insertion member 42 is aligned with the hole in the first phalangeal bone 22. Alternatively, the reception member 42 may be aligned with the first phalangeal bone 22 and the insertion member 42 may be aligned with the first metatarsal bone 14. In addition, referring to FIG. 23, a spacer or washer 294 made from hard materials suitable for implantation into the human body may be positioned about the shaft 90 of the external member 78 (FIG. 6) of each fastener 82 and up against the insertion and reception members 42 and 46. These spacers 294 offset the implant 38 from the bones 14 and 22 allowing unimpeded rotational motion by the members 42 and 46, and prevent osteoblastic cells from attempting to grow bone around the implant 38 and impeding axial rotation of each end of the implant 38. The spacer 294 materials may be coated with suitable materials that resist the growth of bone cells about the spacers 294.

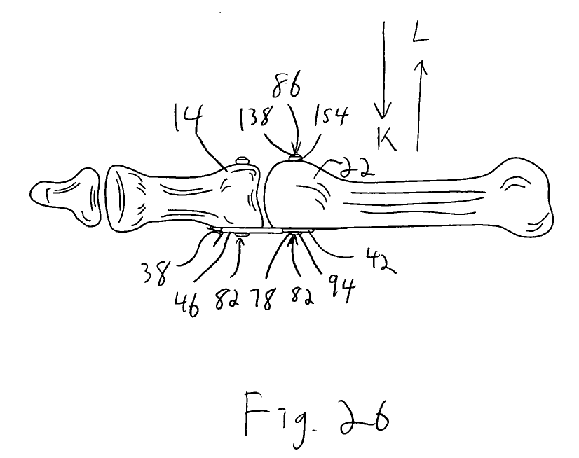

Referring to FIG. 26, the physician then inserts the assembled fasteners 82 into the drilled holes until the spacers 294 (FIG. 23) are in contact with the bones 14 and 22 and the implant 38. For each fastener 82, while holding the head 94 of the external member 78 of one of the fasteners 82, the physician uses the engagement tool 170 (FIG. 13) to pull the internal member 86 in the direction of arrow K until the tabs 122 (FIG. 8) of the internal member 86 have cleared the head 94 of the external member 78.

As the internal member 86 is pulled in the direction of arrow K, the elastomeric component 154 is compressed and extends outwardly to a diameter greater than that of the drilled hole between the head 138 of the internal member 86 and the bone 14 and 22 to prevent the fastener 82 from being pulled in the direction of arrow K out of the hole. The physician then turns the engagement tool 170 (FIG. 13) generally ninety degrees to align and engage the tabs 122 (FIG. 8) with the slot 102 (FIG. 7) of the external member 78. The physician then disengages the engagement tool 170 from the internal member 86. The tabs 122 within the slot 102 of the head 94 of the external member 78 resist the turning motion of the engagement tool 170 and allow the disengagement of the threaded tip 182 (FIG. 13) from the threaded hole 150 (FIG. 8) of internal member 86. The screw cap 166 (FIG. 16) is then screwed to the internal member 86. The physician then repeats the same procedure on the other fastener 82 to secure the implant 38 to the bones 14 and 22 of the joint. The physician then removes the base 194, spacer gauge 202, and spring clamps 198. The physician then pulls the skin and other soft tissue over the implant 38 and sutures the skin.

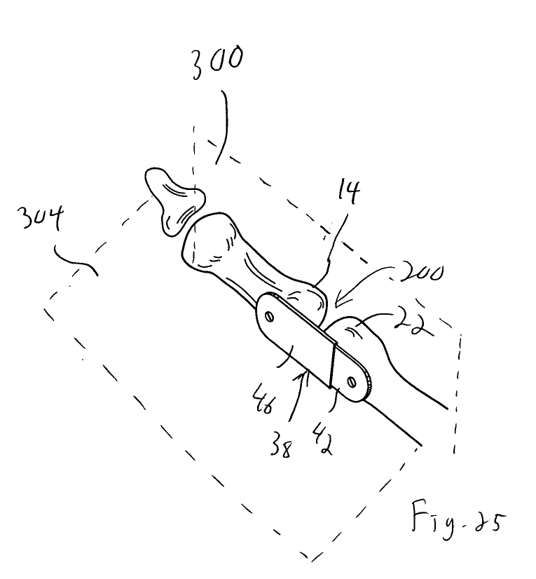

When installed, the mated members 42 and 46 of the implant 38 engage the bones 14 and 22 to prevent each of the bones 14 and 22 from moving out of their generally coplanar alignment in the direction of arrow K or arrow L and thus prevents the bones 14 and 22 from moving back into a deformed bunion or HAV state. Additionally, referring to FIG. 25, the implant 38 expands and contracts as necessary to allow the bones 14 and 22 of the joint 200 to flex and rotate along a sagittal plane 300 relative to each other. When the joint 200 flexes through its range of motion, the relative distance between a point on each bone 14 and 22 increases and decreases due to the natural curvature on each bone head and the change in radius each curvature represents in combination with the other. Because the implant 38 includes two members 42 and 46 that move relative to the bones to which each member is attached and to each other, the implant 38 expands and contracts, or pistons, as the joint 200 moves through its range of motion.

When the joint 200 flexes across its range of motion, the implant 38 expands by way of the insertion member 42 sliding within and away from the reception member 46. Soft connective tissue between the two bones 14 and 22 of the joint 200 provides the elastic response to contract the implant 38 back again by way of the insertion member 42 sliding within and toward the reception member 46 as the range of motion shifts to the other direction. Furthermore, during the sliding of the insertion member 42 within the reception member 46, the distal end 50 (FIG. 5) of the insertion member 42 engages the surgical fastener 82 in the reception member 46 such that the proper minimum distance between the heads of the bones 14 and 22 is maintained as set by the surgical guide 190 (FIG. 17). Thus, the implant 38 prevents the bones 14 and 22 of the joint 200 from moving too close to each other along the plane 300 and compressing the joint 200 and therefore eliminates the need for many of the more complicated conventional bunion surgical procedures.

Patients with HAV can develop a devastating complication following surgery known as Hallux Varus. Hallux Varus is a condition in which the Hallux (big toe) moves medially away from the other toes along a transverse plane 304. Hallux Varus occurs at a rate of 2-17% following bunion correction (Trnka, H. J. et al. acquired hallux varus and clinical tolerability. Foot Ankle Int. 1997; 18:593-597). The implant 38 offers an advantage in that, when implanted to prevent HAV, the implant 38 also prevents Hallux Varus from occurring. During surgery, the MPJ 200 is stabilized in the transverse plane 304, but is allowed normal motion along the frontal and Sagittal plane 300. A result of current surgical procedures for HAV is a tightening of the joint capsule to allow for correction and maintenance of the big toe position. Post-operative motion should be minimized to be successful, however, post operative motion is necessary to prevent the sesmoid bones within the joint 200 from being pulled below the joint 200 and thus locking up the motion of the joint 200. There are several causes for Hallux Varus following HAV surgery, including over-tightening of the medial joint capsule. The implant 38 stabilizes the Hallux (big toe) such that it does not move along the transverse plane 304 but can still move along the Sagittal plane 300, and thus eliminates the need for minimizing postoperative motion of the toe. Because the joint capsule does not need to be tightened, the sesmoid bones do not need to be pulled into a locking position.

The surgical system of the different embodiments of the invention provides numerous benefits. The surgical guide allows the physician to hold the joint stable in its desired location so that holes for the fasteners can be drilled in the precise locations necessary to affix the implant.

Given the potentially large lateral forces against the mated insertion and reception members, the surgical fasteners have to resist pull out. Given the axial rotation of each member about the surgical fastener, the surgical fasteners need to resist walk out. The elastomeric component of the fastener prevents pull out and walk out from the bone while not requiring a nut to secure the distal end of the fastener. The internal and external members used with the elastomeric component allow the fastener to be inserted and secured from one side of the bone and to be easily removed from the bone. The invention thus provides for permanent fastener installation while eliminating pull-out and walk-out. However, if for whatever reason the need arises to remove the fastener, the surgeon can easily release the captured distal end from the proximal end by decompressing the elastomeric component and then remove the fastener. While the fastener is disclosed as a means of preventing pull-out or walk-out of the implant, such a fastener could be employed in many different surgical procedures that currently involve other surgical fasteners.

Another benefit of the invention is that the implant provides strength and structure to the unstable joint under load yet allows the joint to move about the Sagittal plane. It is a further advantage that the invention also allows natural mobility and function of the joint to occur without allowing bones of the joint to move transversely to the Sagittal plane. Also, because the implant prevents the bones from moving transversely to the Sagittal plane, the implant reduces the likelihood that the patient will have to seek later post-operative corrective treatment. It is an additional benefit that the invention utilizes a novel method of affixing a surgical implant to bone in such a way that normal human function will not cause the fastener to pull out of the bone or walk out of the bone. Thus the fastener creates a permanent installation of the implant or permanently holds bones together.

An additional benefit of the application of this invention is that it can permanently decompress a joint, thus alleviating joint compression pain and provide the joint with an unencumbered range of motion. Another advantage is that the compression generated by the fastener can be controlled by altering the durometer and the length of the elastomeric component in relation to the lengths of the internal and external components.

It is an additional benefit of the invention that the physician is provided with a surgical guide device that allows for fast and accurate installation of an implant device during joint surgery. For example, the surgical guide attaches to the bones of the joint and holds them in place. It employs a spacer gauge to set the exact minimum distance between the heads of the bones in the joint. It also provides pilot holes for drilling through the bones and a marking system that indicates what size implant needs to be used for the particular joint being stabilized. Lastly, it stabilizes the joint during the installation of the implant and engagement of the surgical fasteners.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A fastener for use during surgical procedures, comprising:
a first member having a first shaft;
a second member having a second shaft; and
an elastomeric component positioned about at least one of said first and second members, wherein said first shaft of said first member engages and secures said second shaft of said second member thereabout within a bore such that said elastomeric component is compressed between said first and second members and expands to retain said first and second members within the bore.

2. The fastener of claim 1, wherein said first member includes a head at a first end of said first shaft and a tab at a second end of said first shaft and said second member includes a head at a first end of said second shaft, said tab engaging said head of said second member such that said elastomeric component is compressed between said head of said first member and said second shaft of said second member.

3. The fastener of claim 1, wherein said elastomeric component is a compressible ring positioned about said first shaft of said first member.

4. The fastener of claim 1, wherein said second member includes a bore in said second shaft that telescopingly receives said first shaft of said first member as said second member engages said first member.

5. The fastener of claim 1, wherein said first member includes a head at a first end of said first shaft and a threaded hole at a second end of said first shaft, said threaded hole engaging a tool that pulls said head of said first member toward said second shaft of said second member such that said elastomeric member is compressed therebetween.

6. The fastener of claim 1, wherein said first member includes a head at a first end of said first shaft and a tab at a second end of said first shaft and said second member includes a head at a first end of said second shaft, said fastener being inserted in a hole in a bone such said head of said first member extends out of a first end of the hole and said head of said second member extends out of a second end of said hole, said tab engaging said head of said second member such that said elastomeric component expands between said head of said first member and the bone to secure said head of said second member at said second end of the bone.

7. The fastener of claim 1, wherein said first shaft of said first member includes a threaded hole at a first end thereof, said first end extending out of said second shaft of said second member and including a screw for closing said threaded hole.

8. A surgical implant for attachment to two bones at a joint, comprising:
a first member having a first hole and being connected to a first bone by a fastener at said first hole; and
a second member having a second hole and being connected to a second bone by a fastener at said second hole, said second member being configured to receive at least a portion of said first member therein such that said first member slidably moves within said second member as the first and second bones of the joint go through a range of motions.

9. The surgical implant of claim 8, wherein said first member is planar with a flat first end and a curved second end and said second member has a mouth at a first end and a curved second end, said mouth receiving said first end of said first member.

10. The surgical implant of claim 8, wherein said second member includes a bore therein that receives at least a portion of said second member, said first member slidably pistoning within said bore as the first and second bones of the joint go through a range of motions.

11. The surgical implant of claim 8, wherein said first member rotates about the fastener in said first hole and said second member rotates about the fastener in said second hole as the first and second bones of the joint go through a range of motion.

12. The surgical implant of claim 8, wherein said first member has a first end that is received in said second member, said first end engaging the fastener in said second hole of said second member such that the first and second bones of the joint are maintained a minimum distance from each other.

13. The surgical implant of claim 8, wherein said first and second members are slidably engaged along a vertical plane to allow the first and second bones of the joint to move parallel to said vertical plane.

14. The surgical implant of claim 8, wherein said first and second members are slidably engaged along a vertical plane to impede the first and second bones of the joint from moving along a horizontal plane that is transverse to said vertical plane.

15. The surgical implant of claim 8, wherein the fastener includes a first cylindrical member having a first shaft, a second cylindrical member having a second shaft, and an elastomeric component, wherein said first shaft of said first cylindrical member engages and secures said second shaft of said second cylindrical member thereabout within a bore such that said elastomeric component is compressed between said first and second cylindrical members and expands to retain said first and second cylindrical members within the bore.

16. A surgical guide system for drilling at a surgical site for installation of an implant, comprising:
a base member;
at least one clamp for connecting said base member to a surgical site;
a first guide member configured to be moved relative to said base member and including a first drill pilot hole; and
a second guide member configured to be moved relative to said first guide member and including a second drill pilot hole, said first guide member being moved about said base member and secured to a position such that said first drill pilot hole is aligned with a first point at the surgical site and said second guide member being moved about said first guide member to a position such that said second drill pilot hole is aligned with a second point at the surgical site in order to guide a drill into the first and second points.

17. The surgical guide system of claim 16, wherein said base member includes a slot that receives a key connected to said first guide member, said first guide member being movable along said base member by said key.

18. The surgical guide system of claim 16, further including a key received within said base member, said first guide member including a groove receiving said key such that said first guide member may be moved about said key and said key may be locked within said groove to secure said first guide member.

19. The surgical guide system of claim 16, wherein said second guide member includes a leg and said first guide member includes a groove that slidably receives said leg, said second guide member being movable within said groove and locked within said groove to secure said second guide member.

20. The surgical guide system of claim 16, wherein said first drill pilot hole of said first guide member and said second pilot drill hole of said second guide member are aligned along a plane as said second guide member moves with respect to said first guide member.

21. The surgical guide system of claim 16, wherein said second guide member includes markings thereon that indicate the distance between the first pilot drill hole of the first guide member and the second pilot drill hole of the second guide member as said second guide member is moved with respect to said first guide member.

22. The surgical guide system of claim 16, wherein said first and second pilot drill holes are aligned such that first and second drill holes can be drilled into first and second bones, respectively, at the surgical site to attach the implant to first and second bones, the implant including a first member connected to the first drilled hole in the first bone by a fastener and a second member connected to a second drilled hole in the second bone by a fastener, said second member being configured to receive at least a portion of said first member therein such that said first member slidably moves within said second member as the first and second bones of the joint go through a range of motions.

23. A surgical kit for treatment of a joint, comprising:
a surgical guide system for drilling first and second bones at the joint, including a base member configured to be connected at the joint, a first guide member configured to be moved about said base member and including a first drill pilot hole, and a second guide member configured to be moved about said first guide member and including a second drill pilot hole, said first and second guide members being moved such that said first and second drill pilot holes are aligned with a first bone and a second bone at the joint, respectively, to guide a drill into the first and second bones;
a surgical implant for attachment to the two bones at the joint, including a first implant member connected to the first bone and a second implant member connected to the second bone, said second implant member being configured to receive at least a portion of said first implant member therein such that said first implant member slidably moves within said second implant member as the first and second bones go through a range of motions; and
a surgical implant for attachment to the two bones at the joint, including a first member having a first shaft and a second member having a second shaft, and an elastomeric component, wherein said first shaft of said first member is positioned within a drilled hole in the first or second bone and engages and secures said second shaft of said second member thereabout such that said elastomeric component is compressed between said first and second members and expands to retain said first and second members within the hole.

24. A method for surgically treating a joint condition, comprising:
providing a surgical guide having a first guide member with a first pilot hole and a second guide member with a second pilot hole;
connecting the surgical guide to the joint;
moving the first guide member to a position where the first pilot hole is aligned with a first bone of the joint;
moving the second guide member with respect to the first guide member to a position where the second pilot hole is aligned with a second bone of the joint; and
using the first pilot hole to drill a first drill hole into the first bone and using the second pilot hole to drill a second drill hole into the second bone.

25. The method of claim 24, further comprising,
providing an implant having a first implant member with a first hole and a second implant member with a second hole, wherein the second implant member receives at least a portion of the first implant member therein;
providing a plurality of fasteners having a first member, a second member, and an elastomeric component;
providing a surgical spacer for receiving the fastener and being located between the implant and the bone;
for each of the first and second implant members, assembling the first member of a fastener with the elastomeric component and with the second member of a fastener;
inserting each of the assembled fasteners into each of the first and second holes of the first and second implant members, into a surgical spacer, and into each of the first and second drill holes;
pulling the first member of each fastener in a direction out of each drill such that the elastomeric component of each fastener expands between the first and second members of the fastener to secure the fastener within each drill hole;
securing the first member of each fastener to the second member of each fastener such that each elastomeric component retains each fastener within each drill hole and retains each of the first and second implant members to each of the first and second bones wherein the first implant member can move telescopingly within the second implant member.

26. A method for installing a surgical fastener into a drill hole in a bone, comprising:
providing a fastener having a first member, a second member, and an elastomeric component;
assembling the first member of the fastener with the elastomeric component and the second member of the fastener;
inserting the assembled fastener into a drill hole in a bone;
pulling the first member of the fastener in a direction out of the drill hole such that the elastomeric component of the fastener is compressed and expands between the first and second members of the fastener to secure the fastener within the drill hole; and
securing the first member of the fastener to the second member of the fastener such that the compressed elastomeric component retains the fastener within the drill hole.
